(19) United States
(12) Patent Application Publication
(10) Pub. No.: US 2024/0423861 A1
(43) Pub. Date: Dec. 26, 2024

(54) REHABILITATION ASSISTANCE SYSTEM, REHABILITATION ASSISTANCE METHOD, AND PROGRAM

(71) Applicant: TOYOTA JIDOSHA KABUSHIKI KAISHA, Toyota-shi (JP)

(72) Inventors: Takuya IWATA, Toyota-shi (JP); Makoto KOBAYASHI, Nisshin-shi (JP)

(73) Assignee: TOYOTA JIDOSHA KABUSHIKI KAISHA, Toyota-shi (JP)

(21) Appl. No.: 18/652,032

(22) Filed: May 1, 2024

(30) Foreign Application Priority Data
Jun. 23, 2023 (JP) 2023-103452

(51) Int. Cl. A61H 1/02; G16H 20/30

(52) U.S. Cl. CPC A61H 1/02; G16H 20/30; A61H 2201/5043

(57) ABSTRACT

The rehabilitation assistance system includes: a goal acquisition unit configured to acquire a long-term goal to be achieved over a long period of time, the long-term goal having a plurality of achievement stages for recovering functions of a predetermined physical action of a rehabilitation patient, and a short-term goal to be achieved in a short period of time which is shorter than the long period of time, the short-term goal having a plurality of achievement stages; a practice result acquisition unit configured to acquire a practice item practiced on each practice day as practice results; an achievement stage acquisition unit configured to acquire the achievement stage achieved by the practice results; a prediction unit configured to predict, based on the achieved achievement stage; and a display unit configured to cause a display device to display the practice results.

### CROSS REFERENCE TO RELATED APPLICATIONS

This application is based upon and claims the benefit of priority from Japanese patent application No. 2023-103452, filed on Jun. 23, 2023, the disclosure of which is incorporated herein in its entirety by reference.

### BACKGROUND

The present disclosure relates to a rehabilitation assistance system, a rehabilitation assistance method, and a program.

Japanese Patent No. 6355073 discloses a technique for visualizing and presenting progress and results of training such as rehabilitation (which may be simply referred to as rehab).

### SUMMARY

It may sometimes be difficult for a person who performs training such as rehabilitation to maintain his/her motivation for performing the training as the period of time from the start of the training to the time of discharge may be a long one. Therefore, a system that enables a trainee or medical staff to set a more precise goal for the training or to obtain information that is necessary for the medical staff to give advice having a long-term perspective regarding the training to the trainee, thereby highly motivating the trainee to continue training, is needed. However, there is currently no related art which includes a mechanism for enabling a trainee or medical staff to recognize a goal or a mechanism for checking the progress of the training for achieving the goal. Even when data of training results is extracted by machine learning or the like, it is difficult to construct a mechanism related to a motivation.

The present disclosure has been made in order to solve the aforementioned problem and an aim of the present disclosure is to provide a rehabilitation assistance system, a rehabilitation assistance method, and a program capable of enabling a rehabilitation patient or medical staff to intuitively check and recognize delay to a goal or a progress state.

A rehabilitation assistance system according to one aspect of the present disclosure includes: a goal acquisition unit configured to acquire a long-term goal to be achieved over a long period of time, the long-term goal having a plurality of achievement stages for recovering functions of a predetermined physical action of a rehabilitation patient, and a short-term goal to be achieved in a short period of time which is shorter than the long period of time, the short-term goal having a plurality of achievement stages; a practice result acquisition unit configured to acquire a practice item practiced on each practice day as practice results; an achievement stage acquisition unit configured to acquire the achievement stage achieved by the practice results; a prediction unit configured to predict, based on the achieved achievement stage, estimated dates of goal achievement including an estimated date of achievement of the long-term goal and an estimated date of achievement of the short-term goal; and a display unit configured to cause a display device to display the practice results, the achievement stage, and the estimated dates of goal achievement in a superimposed manner.

The rehabilitation assistance system may further include a storage unit configured to store the practice results, the achievement stage, and the estimated dates of goal achievement.

In the rehabilitation assistance system, the storage unit may store the practice results, the achievement stage, and dates of goal achievement including the date of achievement of the short-term goal and the date of achievement of the long-term goal of another rehabilitation patient, and the prediction unit may predict the estimated dates of goal achievement based on the practice results, the achievement stage, and the dates of goal achievement of the other rehabilitation patient.

The rehabilitation assistance system may further include a practice item acquisition unit configured to acquire the practice items, and the practice item acquisition unit may acquire the practice items based on the practice results, the achievement stage, and the dates of goal achievement of the other rehabilitation patient, and the estimated dates of goal achievement predicted by the prediction unit.

In the rehabilitation assistance system, the storage unit may store an assistant who has assisted the rehabilitation patient when the rehabilitation patient has performed the practice items for recovering his/her functions of the physical action in association with the practice results.

In the rehabilitation assistance system, the assistant may include an attribute of at least one of a physical therapist, an occupational therapist, a speech therapist, a doctor, or a nurse, and the storage unit may store each attribute in association with the practice results.

In the rehabilitation assistance system, the display unit may extract the practice results associated with the specific assistant and cause a display device to display the extracted practice results.

In the rehabilitation assistance system, the prediction unit may predict each estimated date of goal achievement based on the practice results associated with each assistant.

In the rehabilitation assistance system, the storage unit may store an individual practice carried out by one person or a group practice carried out by a plurality of persons when the rehabilitation patient practices the practice items for recovering his/her functions of the physical action in association with the practice results.

A rehabilitation assistance method according to one aspect of the present disclosure includes: acquiring a long-term goal to be achieved over a long period of time, the long-term goal having a plurality of achievement stages for recovering functions of a predetermined physical action of a rehabilitation patient, and a short-term goal to be achieved in a short period of time which is shorter than the long period of time, the short-term goal having a plurality of achievement stages; acquiring practice items practiced on each practice day as practice results; acquiring the achievement stage achieved by the practice results; predicting estimated dates of goal achievement including an estimated date of achievement of the long-term goal and an estimated date of achievement of the short-term goal based on the achieved achievement stage; and causing a display device to display the practice results, the achievement stage, and the estimated dates of goal achievement in a superimposed manner.

The rehabilitation assistance method may include storing the practice results, the achievement stage, and the estimated dates of goal achievement in a storage unit.

The rehabilitation assistance method may include: storing the practice results, the achievement stage, and dates of goal achievement including the date of achievement of the short-term goal and the date of achievement of the long-term goal of another rehabilitation patient; and predicting, when the estimated dates of goal achievement are predicted, the estimated dates of goal achievement based on the practice results, the achievement stage, and the dates of goal achievement of the other rehabilitation patient.

The rehabilitation assistance method may include: acquiring the practice items; and acquiring, when the practice items are acquired, the practice items based on the practice results, the achievement stage, and the dates of goal achievement of the other rehabilitation patient, and the estimated dates of goal achievement that have been predicted.

The rehabilitation assistance method may include storing, when the practice results, the achievement stage, and the estimated dates of goal achievement are stored, an assistant who has assisted the rehabilitation patient when the rehabilitation patient has performed the practice items for recovering his/her functions of the physical action in association with the practice results.

In the rehabilitation assistance method, the assistant may include an attribute of at least one of a physical therapist, an occupational therapist, a speech therapist, a doctor, or a nurse, and when the practice results, the achievement stage, and the estimated dates of goal achievement are stored, each attribute may be stored in association with the practice results.

In the rehabilitation assistance method, when the practice results, the achievement stage, and the estimated dates of goal achievement are displayed on the display device in a superimposed manner, the practice results associated with the specific assistant may be extracted and the display device may display these extracted practice results.

The rehabilitation assistance method may include predicting, when the estimated dates of goal achievement are predicted, each estimated date of goal achievement based on the practice results associated with each assistant.

The rehabilitation assistance method may include storing, when the practice results, the achievement stage, and the estimated dates of goal achievement are stored, an individual practice carried out by one person or a group practice carried out by a plurality of persons when the rehabilitation patient practices the practice items for recovering his/her functions of the physical action in association with the practice results.

A program according to one aspect of the present disclosure causes a computer to execute processing of: acquiring a long-term goal to be achieved over a long period of time, the long-term goal having a plurality of achievement stages for recovering functions of a predetermined physical action of a rehabilitation patient, and a short-term goal to be achieved in a short period of time which is shorter than the long period of time, the short-term goal having a plurality of achievement stages; acquiring practice items practiced on each practice day as practice results; acquiring the achievement stage achieved by the practice results; predicting estimated dates of goal achievement including an estimated date of achievement of the long-term goal and an estimated date of achievement of the short-term goal based on the achieved achievement stage; and causing a display device to display the practice results, the achievement stage, and the estimated dates of goal achievement in a superimposed manner.

According to the present disclosure, it is possible to provide a rehabilitation assistance system, a rehabilitation assistance method, and a program capable of enabling a rehabilitation patient or medical staff to intuitively check and recognize delay to a goal or a progress state.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a configuration diagram illustrating an assistance system according to a first embodiment;

FIG. 2 is a block diagram illustrating a rehabilitation assistance apparatus according to the first embodiment;

FIG. 3 is a diagram illustrating rehabilitation patient information according to the first embodiment;

FIG. 4 is a diagram illustrating evaluation items for evaluating a level of recovery of a rehabilitation patient according to the first embodiment;

FIG. 5 is a diagram illustrating a display screen that displays goals set for the rehabilitation patient according to the first embodiment;

FIG. 6 is a graph illustrating practice results and FIM of a target person according to the first embodiment, in which a horizontal axis indicates the number of days and two vertical axes indicate practice hours and the FIM;

FIG. 7 is a graph illustrating practice results and FIM of a past patient according to the first embodiment, in which a horizontal axis indicates the number of days and two vertical axes indicate practice hours and the FIM;

FIG. 8 is a graph illustrating practice results and FIM of the past patient and the target patient according to the first embodiment, in which a horizontal axis indicates the number of days and two vertical axes indicate practice hours and the FIM;

FIG. 9 is a diagram illustrating a display screen in which a display unit displays practice results according to the first embodiment;

FIG. 10 is a diagram illustrating a display screen in which the display unit displays a short-term goal, a long-term goal, practice results, and an achievement stage according to the first embodiment;

FIG. 11 is a diagram illustrating a display screen in which the display unit displays a short-term goal, a long-term goal, practice results, an achievement stage, and estimated dates of goal achievement according to the first embodiment;

FIG. 12 is a flowchart illustrating a rehabilitation assistance method according to the first embodiment; and

FIG. 13 is a block diagram showing a configuration example of the rehabilitation assistance apparatus according to the first embodiment.

### DESCRIPTION OF EMBODIMENTS

Hereinafter, with reference to the drawings, a specific configuration of this embodiment will be described. The following description indicates a preferable embodiment of the present disclosure and the scope of the present disclosure is not limited to the following embodiment. Further, not all the structures explained in the embodiment may be necessary as means for solving the problem. For a purpose of clarifying the description, the following description and the drawings will be omitted and simplified as appropriate. Throughout the drawings, the same components are denoted by the same reference symbols and overlapping descriptions will be omitted as necessary.

### First Embodiment

A rehabilitation training system according to this embodiment performs assistance for enabling a trainee to carry out training for the purpose of recovering functions of his/her physical actions. The training includes one or a plurality of kinds of training items. The training items are also referred to as practice items. The trainee here is, for example, a person suffering from paralysis in a part of his/her body which is caused by a stroke due to cerebrovascular disease, or a spinal cord injury. The trainee may be any person who performs training to recover functions of his/her physical actions and is not limited to a person suffering from paralysis in a part of his/her body. The trainee is also referred to as a rehabilitation patient. The physical action functions of the rehabilitation patient have been impaired due to the above disease. In this embodiment, physical actions are, for example, actions included in Activities of Daily Living (ADL) and include actions such as eating, toileting, dressing, walking, etc.

A rehabilitation patient carries out training including one or a plurality of kinds of practice items in order to recover the functions of his/her impaired physical actions. To be specific, for example, in order to recover functions of a physical action eating, the rehabilitation patient carries out a practice item of a meal intake action, a practice item of a swallowing action, and so on. Further, in order to recover functions of the toileting action, for example, the rehabilitation patient carries out a practice item of a moving action for moving to each of a bed, a wheelchair, and a toilet seat, a practice item for toileting, and so on. Further, in order to recover the function of the walking action, for example, the rehabilitation patient carries out a practice item for maintaining the sitting posture, a practice item for maintaining the standing posture, a practice item of flat ground walking, a practice item of walking on a slope, and so on. Further, the practice items include, besides the ones in which the rehabilitation patient actively carries out physical actions as described above, passive ones such as a massage in which the rehabilitation patient receives stimulation to his/her joints or muscles from an assistant. The aforementioned training including the practice items is also referred to as rehabilitation, a rehabilitation training, or rehab.

Some training performed using the rehabilitation assistance system may require assistance by therapists. Therapists are persons who have expertise that is necessary to assist rehabilitation patients. In this embodiment, the therapists include a physical therapist, an occupational therapist, and a speech therapist. The therapists assist training performed by the rehabilitation patients in accordance with expertise that they each have.

The physical therapist is also referred to as a PT. The physical therapist is a person who offers a physical therapy for persons who have physical disabilities mainly for the purpose of recovering fundamental action abilities. The physical therapy is a therapeutic treatment performed on persons whose functions of physical actions have been impaired due to diseases, injuries, disabilities, and so on for the purpose of maintaining or improving these functions. The physical therapist carries out a physical therapy by using exercises, heat, electricity, and so on.

The occupational therapist is also referred to as an OT. The occupational therapist is a person who offers an occupational therapy for persons who have physical or mental disabilities mainly for the purpose of recovering their applicative action abilities or social adaptability. The “occupation” here includes daily activities such as bathing, dressing, and toileting.

The speech therapist is also referred to as an ST. The speech therapist is a person who carries out language training and other training exercises and provides tests, advice, guidance, and other assistance necessary for the above training exercises for persons who have disabilities in their speech functions, language functions, or hearing for the purpose of maintaining and improving these functions. The speech therapist further conducts training items such as meal intake or swallowing under instructions of doctors or dentists.

Hereinafter, persons such as the PT, the OT, and the ST who assist rehabilitation training performed by rehabilitation patients are also called assistants. The assistants may include doctors, nurses and so on. The assistant assists a rehabilitation patient when the rehabilitation patient practices the practice items for recovering functions of his/her physical actions.

In the following, first, an <assistance system> including a rehabilitation assistance system according to this embodiment will be described. After that, a <training apparatus>, a <terminal>, and a <rehabilitation assistance apparatus> will be described. Then, a <patient information acquisition unit>, a <goal acquisition unit>, a <practice item acquisition unit>, a <practice result acquisition unit>, an <achievement stage acquisition unit>, a <prediction unit>, a <storage unit>, and a <display unit>, which are configurations of the rehabilitation assistance apparatus, will be described, and then a <rehabilitation assistance method> will be described.

### <Assistance System>

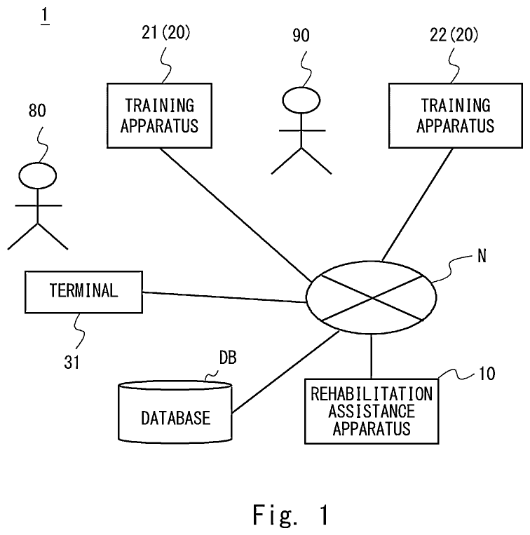

FIG. 1 is a configuration diagram illustrating an assistance system according to the first embodiment. An assistance system 1 includes a rehabilitation assistance apparatus 10, training apparatuses 21 and 22, a terminal 31, and a database DB. While the assistance system 1 includes two training apparatuses 21 and 22 in FIG. 1, the assistance system 1 may include only one training apparatus 21 or may include three or more training apparatuses 20. The training apparatuses 21 and 22 are collectively referred to as a training apparatus 20. Further, the assistance system 1 may not include the training apparatus 20 when the training apparatus 20 is not necessary. A plurality of rehabilitation patients 90 may use the assistance system 1.

### <Training Apparatus>

The training apparatus 20 is, for example, training equipment for providing walking practice for improving walking functions. Accordingly, a rehabilitation patient 90 performs rehabilitation training for improving his/her walking functions. The training equipment for providing walking practice is, for example, Welwalk WW-1000 or WW-2000. As a matter of course, the rehabilitation training is not limited to walking practice and may instead be training for meal intake action or toileting. Therefore, the training apparatus 20 may be training equipment for enabling the rehabilitation patient 90 to perform a meal intake action or toileting. The training apparatus 20 may include drive equipment such as a motor, a sensor, and so on. The training apparatus 20 is installed in a rehabilitation facility of a hospital or the like. As a matter of course, the training apparatus 20 may be equipment installed in the house of the rehabilitation patient 90.

### <Terminal>

The terminal 31 is a device that enables at least one of an assistant 80 or the rehabilitation patient 90 (hereinafter each of them will be referred to as an assistant 80 or the like) to enter data. Further, the terminal 31 is a device that can output data output from the rehabilitation assistance apparatus 10. The terminal 31 is, for example, a tablet computer, a smartphone, a personal computer or the like. The terminal 31 inputs data by an input device such as a keyboard, a mouse, a touch panel, a microphone or the like. The terminal 31 outputs data by an output device including a display device such as a display, a printer or the like. The assistant 80 or the like can input and output data regarding the rehabilitation patient 90 or rehabilitation training performed by the rehabilitation patient 90 using the terminal 31. The terminal 31 may be a device attached to the training apparatus 20 or may be a device attached to the rehabilitation assistance apparatus 10. The assistance system 1 may include a plurality of terminals 31.

### <Rehabilitation Assistance Apparatus>

The rehabilitation assistance apparatus 10 is a device to/from which data can be input/output. The rehabilitation assistance apparatus 10 itself may be referred to as a rehabilitation assistance system, or a combination of the rehabilitation assistance apparatus 10 with at least one of the training apparatus 20, the terminal 31, or the database DB may be referred to as a rehabilitation assistance system. The rehabilitation assistance apparatus 10 serves as a rehabilitation assistance system which assists rehabilitation training. The rehabilitation assistance apparatus 10 may be implemented by a computer including a memory, a processor, a display device and the like.

The rehabilitation assistance apparatus 10 may acquire data from the training apparatus 20, the terminal 31, and the database DB. Further, the rehabilitation assistance apparatus 10 may output data to the training apparatus 20, the terminal 31, and the database DB. The training apparatus 20, the terminal 31, the database DB, and the rehabilitation assistance apparatus 10 may be connected to one another in such a way that they can communicate with one another via a network N. The network N is a communication line network such as the Internet, an intranet, a mobile telephone network, or a Local Area Network (LAN).

The acquisition and the output of data by the rehabilitation assistance apparatus 10 are not limited to the ones that use the network N. For example, at least a part of data may be acquired and output via a USB memory or the like. The data may be acquired and output by a wireless communication line such as WiFi (registered trademark) or by a wired communication line. The rehabilitation assistance apparatus 10 may acquire and output data from and to the training apparatus 20, the terminal 31, and the database DB via at least one of wireless or wired communication line. The rehabilitation assistance apparatus 10 includes an interface for wireless or wired data input/output. The acquisition of data by the rehabilitation assistance apparatus 10 may be performed by the assistant 80 or the like who selects necessary data and write the data into the rehabilitation assistance apparatus 10, or may be automatically performed.

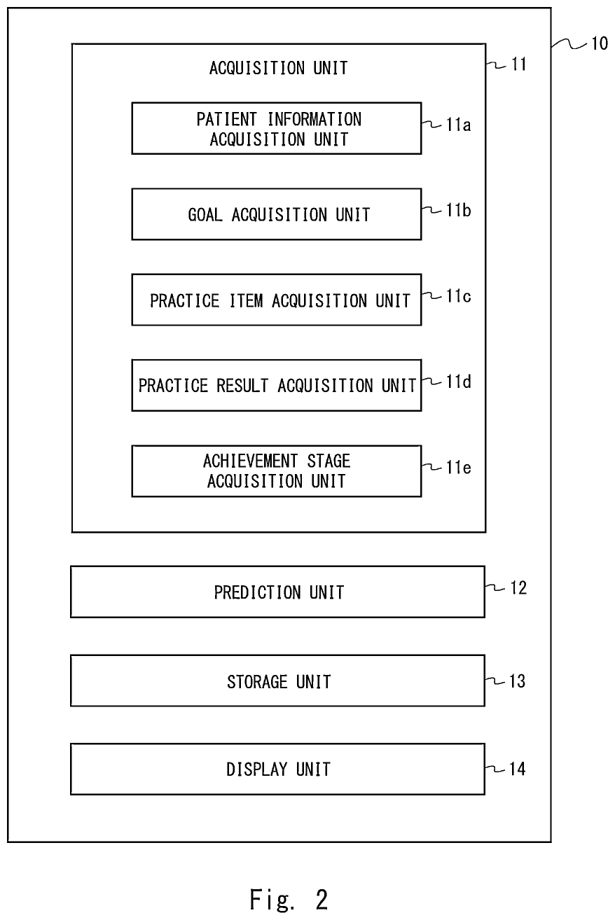

FIG. 2 is a block diagram illustrating the rehabilitation assistance apparatus 10 according to the first embodiment. As shown in FIG. 2, the rehabilitation assistance apparatus 10 includes an acquisition unit 11, a prediction unit 12, a storage unit 13, and a display unit 14. The acquisition unit 11 may include a patient information acquisition unit 11a, a goal acquisition unit 11b, a practice item acquisition unit 11c, a practice result acquisition unit 11d, and an achievement stage acquisition unit 11e.

Like the terminal 31, the rehabilitation assistance apparatus 10 may be a tablet computer, a smartphone, a personal computer or the like that includes an input device and a display device. The rehabilitation assistance apparatus 10 may use some of the functions of the terminal 31. Further, the rehabilitation assistance apparatus 10 is not limited to a physically single apparatus. For example, the storage unit 13 may be a database DB provided separately from the main body apparatus.

### <Patient Information Acquisition Unit>

The patient information acquisition unit 11a acquires rehabilitation patient information regarding the rehabilitation patient 90. The patient information acquisition unit 11a may acquire rehabilitation patient information from at least one of the storage unit 13 or the database DB (each of them will be referred to as a storage unit 13 or the like). The rehabilitation patient information may include attributes and so on of the rehabilitation patient 90. The patient information acquisition unit 11a may acquire rehabilitation patient information of other rehabilitation patients 90 (including a past rehabilitation patient 90).

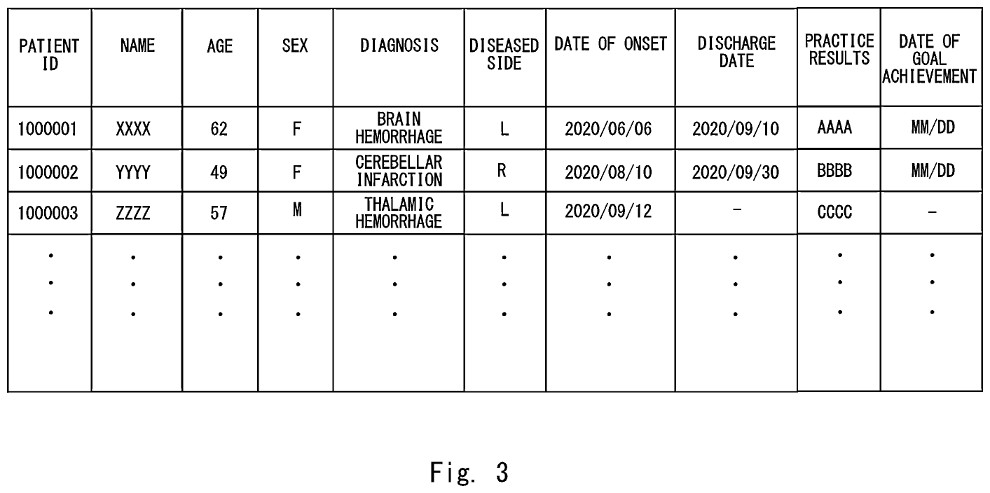

FIG. 3 is a diagram illustrating the rehabilitation patient information according to the first embodiment. As shown in FIG. 3, the rehabilitation patient information includes the patient ID, the name, the age, the sex, the diagnosis, the diseased side, the date of the onset of symptoms, the discharge date, practice results, the date of goal achievement, etc. The diseased side, which is data indicating the diseased leg that is paralyzed, is shown by a left leg (L) or a right leg (R). As shown in the patient ID1000003 in FIG. 3, the discharge date is left blank for a rehabilitation patient 90 who has not been discharged, i.e., an inpatient rehabilitation patient 90. In other words, it is possible to identify whether or not the rehabilitation patient 90 is in the hospital or has been discharged by checking whether the discharge date has been entered.

The rehabilitation patient information may be stored in the storage unit 13 or the like as the assistant 80 or the like inputs, for example, each of the items through the terminal 31. Further, the rehabilitation patient information may be checked by the assistant 80 or the like who enters the patient ID or the name through the terminal 31. Further, when the rehabilitation assistance apparatus 10 acquires data from a plurality of hospitals or facilities, a facility ID indicating the hospital or the facility where the rehabilitation training has been performed may be added.

As a matter of course, the items shown in FIG. 3 are examples of the rehabilitation patient information and some of the items may be omitted. Further, items that are not shown in FIG. 3 may be added. Specifically, the rehabilitation patient information may include practice items, practice results, an achievement stage, a short-term goal, a long-term goal, an estimated date of achievement of the short-term goal, an estimated date of achievement of the long-term goal, a date of achievement of the short-term goal, and a date of achievement of the long-term goal that will be described later. Further, as the rehabilitation patient information, data of higher-order brain functions, the current Functional Independence Measure (FIM), the current adaptive equipment, the current assistive device, and a suspending device may be associated with the patient ID. The FIM, which is also referred to as a functional independence measure, defines one of evaluation methods for ADL. The FIM may include rate on a 7-point scale from one point to seven points based on the level of assistance.

Further, the rehabilitation patient information may include the physical size (body height, weight, etc.), the Brunnstrom Recovery Stage (Br. stage), the Stroke Impairment Assessment Set (SIAS), the initial gait FIM, the latest gait FIM, etc. The initial gait FIM is the gait FIM at the time of the onset of symptoms or before rehabilitation training is started. The rehabilitation patient information may include various kinds of data indicating the physical ability of the rehabilitation patient 90, such as evaluation items for evaluating the level of recovery of the rehabilitation patient 90.

The Br. stage is data in which the process of recovery from hemiplegia is divided into six stages based on observation. The Br. stage may contain the items in the lower extremity section, which are main items related to the walking training apparatus. The SIAS is an index for comprehensively evaluating the dysfunction caused by a stroke. The SIAS may contain hip-flexion test (Hip-Flex), knee-extension test (Knee-Ext), and foot-pat test (Foot-Pat). The SIAS may also contain lower extremity light touch (Touch L/E), lower extremity position sense (Position L/E), abdominal muscle strength (Abdominal), and verticality test (Verticality).

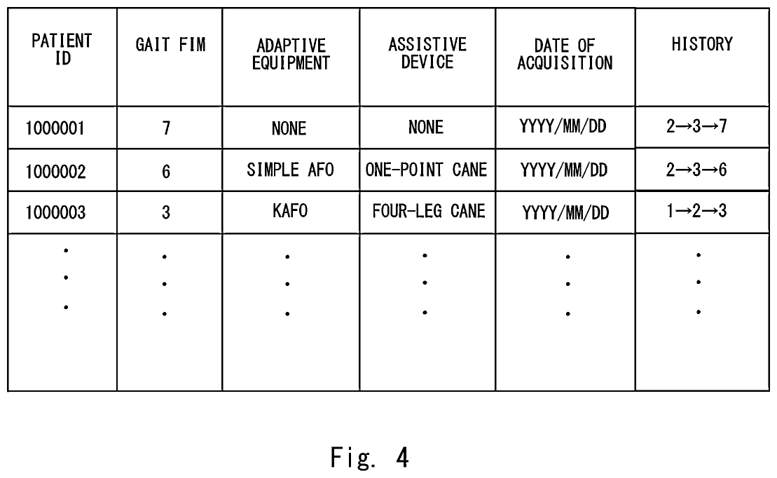

FIG. 4 is a diagram illustrating evaluation items for evaluating the level of recovery of the rehabilitation patient 90 according to the first embodiment. As shown in FIG. 4, regarding walking training, the evaluation items may include three items, namely, gait FIM, adaptive equipment, and an assistive device. An evaluation index indicating the level of recovery is set for each of the evaluation items. Each of the evaluation items may be associated with the date of acquisition when each of the evaluation indices has been acquired and the history of the evaluation indices (in FIG. 4, history of FIM). The storage unit 13 or the like may store the evaluation indices indicating the level of recovery for each of the rehabilitation patients 90.

For example, the gait FIM serves as a general index indicating the level of recovery. Further, the gait FIM is an index indicating the action ability (i.e., walking ability) of the rehabilitation patient 90 when an actuator is not used. When a rehabilitation patient 90 is able to walk 50 m or more without being helped by the assistant 80 and without a harness (assistive device), the highest point, that is, seven points are counted. When the rehabilitation patient 90 can barely walk less than 15 m no matter how hard one assistant 80 assists him/her, the lowest point, that is, one point is counted. Further, when the rehabilitation patient 90 is able to move 50 m with a minimum amount of assistance (amount of assistance: 25% or less), four points are counted. When the rehabilitation patient 90 is able to move 50 m with a moderate level of assistance (amount of assistance: 25% or more), three points are counted. Accordingly, as the recovery progresses, the gait FIM of the rehabilitation patient 90 is gradually increased.

The evaluation index of the gait FIM is indicated by seven levels since it is input using integers from one to seven. The evaluation index of the gait FIM may instead be indicated by eight levels, including a case in which no data is input (i.e., blank).

The adaptive equipment complements or replaces the physical functions of the rehabilitation patient 90 and is used continuously over a long period of time. The rehabilitation patient 90 walks wearing the adaptive equipment. The item of the adaptive equipment is indicated by nine types, namely, none, others, a simple Ankle Foot Orthoses (AFO), with PAFO chair, no PAFO chair, Remodeled Adjustable Posterior Strut (RAPS)-AFO, Molded (M)-AFO, Knee Ankle Foot Orthoses (KAFO), and blank. That is, the evaluation item of the adaptive equipment is categorized into nine evaluation indices in accordance with the level of recovery.

The assistive device is equipment for assisting walking by the rehabilitation patient 90. The rehabilitation patient 90 walks holding an assistive device such as a cane. The evaluation item of the assistive device is indicated by nine types, namely, none, others, a one-point cane, a four-point cane (small), a four-point cane (large), a four-leg cane, a walker, parallel bars, and blank.

The rehabilitation patient 90 is able to walk using an appropriate assistive device and appropriate adaptive equipment. Further, the assistant 80 is able to recommend an appropriate assistive device and an appropriate adaptive equipment to the rehabilitation patient 90 in accordance with the walking functions of the rehabilitation patient 90. The assistive device and the adaptive equipment that are used are gradually changed in accordance with the level of recovery of the rehabilitation patient 90. In other words, the adaptive equipment and the assistive device are evaluation indices for evaluating the level of recovery of the rehabilitation patient 90.

The rehabilitation patient information may include evaluation indices of all the evaluation items. As a matter of course, regarding some evaluation items, the rehabilitation patient information may be blank. The evaluation indices may be associated with the acquired date of acquisition. Further, the evaluation indices may be associated with the number of days passed since the date of the onset of symptoms and the date of the start of the rehabilitation.

### <Goal Acquisition Unit>

The goal acquisition unit 11b acquires goals to be achieved by the rehabilitation patient 90 who practices practice items. The goals include a long-term goal to be achieved over a long period of time and a short-term goal to be achieved in a short period of time. The short period of time is a period shorter than the long period of time. The long-term goal and the short-term goal may each include a plurality of achievement stages. The rehabilitation patient 90 achieves the short-term goal and the long-term goal after going through the plurality of achievement stages. In this way, the goal acquisition unit 11b acquires a long-term goal to be achieved over a long period of time, the long-term goal having a plurality of achievement stages for recovering functions of predetermined physical actions of the rehabilitation patient 90, and a short-term goal to be achieved in a short period of time shorter than the long period of time, the short-term goal having a plurality of achievement stages.

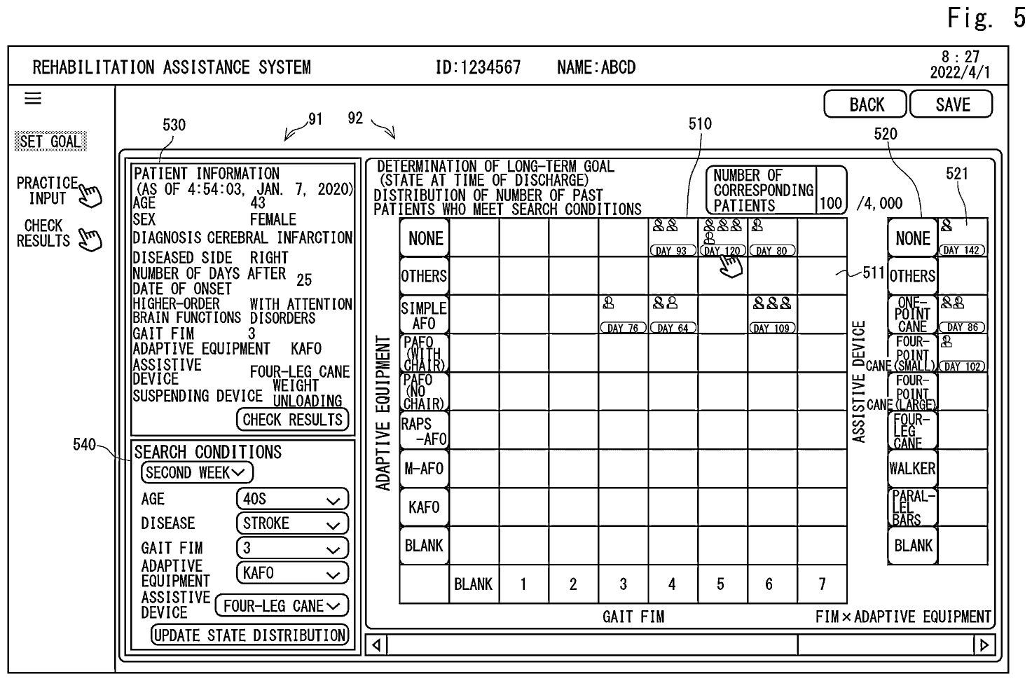

FIG. 5 is a diagram illustrating a display screen that displays goals set for the rehabilitation patient according to the first embodiment. As shown in FIG. 5, for example, the display unit 14 of the rehabilitation assistance apparatus 10 may cause a display device to display rehabilitation patient information of a past rehabilitation patient 90 in order to set the goals for the rehabilitation patient 90.

The display screen includes a first display region 510, a second display region 520, a patient information display region 530, and a search condition display region 540. The patient information display region 530 displays patient information regarding the rehabilitation patient 90 (this patient is also referred to as a target patient 91) for which the goal is set. The patient information display region 530 is, for example, a window that displays the age, the sex, the diagnosis, the diseased side and the like of the target patient 91.

The search condition display region 540 displays search conditions for looking for a past rehabilitation patient 90 who is similar to the target patient 91. The past rehabilitation patient 90 is called a past patient 92. That is, the search condition display region 540 is a window for enabling the assistant 80 or the like to enter search conditions. The search condition display region 540 displays pull-down menus for enabling the assistant 80 or the like to input search conditions.

In FIG. 5, the age (forties), the disease (stroke), the gait FIM (3), the adaptive equipment (KAFO), the assistive device (four-leg cane) and the like are set as the search conditions. The data of the search conditions is input in accordance with the information on the target patient 91. Further, the data of the search conditions may be automatically input from the rehabilitation patient information on the target patient 91 or may be manually input by the assistant 80 or the like.

Further, the number of days elapsed since the onset of symptoms (second week) is set as one of the search conditions. It is possible to extract rehabilitation patients similar to the target patient by referring, for example, to data of each rehabilitation patient in the second week from the start of rehabilitation. By clicking a state distribution update distribution button, which is a search button, the rehabilitation assistance apparatus 10 refers to the storage unit 13 or the like and extracts rehabilitation patients 90 who meet the search conditions. Accordingly, the past patients 92 similar to the target patient 91 are extracted from among the past rehabilitation patients 92. It is possible to filter the past patients 92 according to the search conditions in accordance with the target patient 91. It is therefore possible to set the past patients 92 with preconditions similar to those of the target patient 91 as a population.

The first display region 510 displays a state of progress of the target patient 91 so that the assistant 80 or the target patient 91 can grasp the state of progress of the target patient 91. Further, the display in the first display region 510 is updated in accordance with the progress information of the rehabilitation training. For example, the first display region 510 displays information indicating the states of the past patients 92 at the time of discharge who have met the search conditions in order to set the goal at the time of discharge.

The display unit 14 displays, on the display screen, a plurality of past patients 92 divided into groups in accordance with the states of past patients 92 similar to the target patient 91 at the time of discharge. That is, a plurality of past patients 92 are divided into a plurality of groups. Then the first display region 510 indicates the distribution of the numbers of past patients 92 who belong to the respective groups. In this example, the first display region 510 displays the information on the number of past patients 92 who meet the search conditions in a form of a two-dimensional matrix.

The two-dimensional matrix is generated in accordance with the evaluation indices of the evaluation items. Specifically, the horizontal axis of the two-dimensional matrix indicates the gait FIM at the time of discharge and the vertical axis indicates the adaptive equipment at the time of discharge. The evaluation items used to generate the two-dimensional matrix in the first display region 510 are set as priority evaluation items. That is, the gait FIM and the adaptive equipment are priority evaluation items. The evaluation items other than the priority evaluation items are non-priority evaluation items. The non-priority items include the assistive device. Whether one evaluation item is a priority evaluation item or a non-priority evaluation item may be set in advance. That is, the evaluation items that are regarded to be important and indicate the recovery in the rehabilitation training are set as the priority evaluation items. That is, the evaluation items with a high contribution rate to the recovery are set as the priority evaluation items.

The gait FIM is divided into eight levels, including blank, and the adaptive equipment is divided into nine types. Since the past patients 92 are divided into 72 (=8×9) groups, 72 display frames 511 are shown in the first display region 510 in FIG. 5. Each display frame 511 has a rectangular frame.

The display frames 511 display information on the numbers of persons indicating the numbers of past patients 92 who belong to the respective groups. In this example, the number of persons is indicated by the number of icons, the sizes of the icons and the like. For example, one icon indicates 100 persons. Fractions of less than 100 persons are indicated by the size of the icon or the like. A display frame 511 with no icon indicates a group with no past patients 92 similar to the target patient 91. As a matter of course, the information on the number of persons is not limited to the number of icons and the sizes of the icons. The information on the number of persons may be indicated by display color or color shades. The display unit 14 may set a color bar in accordance with the number of persons and change the display color in accordance with the increase/decrease in the number of persons. Alternatively, the display unit 14 may change grayscale shades depending on the number of persons. Further, the display unit 14 may display the number indicating the number of persons in the display frame 511 as the information on the number of persons.

The display frame 511 may further display information on the number of days in accordance with the number of days until discharge. That is, the average value or the median value of the number of days it took for a plurality of past patients 92 who belong to the respective groups to be discharged is the information on the number of days. The target acquisition unit 11b is able to obtain the information on the number of days from the date of admission and the date of discharge of each rehabilitation patient 90. The target acquisition unit 11b refers to the storage unit 13 or the like and calculates the information on the number of persons and the information on the number of days. Whether or not one patient has been discharged can be determined in accordance with the presence or the absence of the data of the discharge date shown in FIG. 3. Then the evaluation indices on the acquisition date that is just before the discharge date are the evaluation indices at the time of discharge.

As described above, the display unit 14 displays information indicating the state of progress of the target patient 91 in 72 groups. Further, the display unit 14 updates and displays information in accordance with the progress of the rehabilitation. The assistant 80 or the like is able to easily grasp the degree of recovery of past patients 92 similar to the target patient 91 at the time of discharge. Further, the assistant 80 or the like is able to grasp how many days from the date of admission to the hospital it took for the past patients 92 similar to the target patient 91 to be discharged from the hospital. The assistant 80 is therefore able to make an appropriate plan for the target patient 91. That is, the assistant 80 is able to set the goal of the rehabilitation training by referring to the recovery processes of the past patients 92. Thus the target patient 91 is able to maintain high motivation for the rehabilitation training.

The assistant 80 or the target patient 91 is able to select a target group. When the assistant 80 clicks the display frame 511 of the group with the largest number of persons or the group with a relatively large number of persons, this group is set as the selected group. Alternatively, a group according to the level which the target patient 91 desires to reach may be specified as the selected group. The assistant 80 is able to refer to a past rehabilitation history of the past patients 92 who belong to the selected group and make a rehabilitation plan regarding the target patient 91. The assistant 80 is able to set a goal based on the comparison between the target patient 91 and the past patients 92 similar to the target patient 91.

Further, a distribution of the number of past patients 92 who belong to the selected group is displayed in the second display region 520. In this example, the display unit 14 displays the past patients 92 in the selected group divided into subgroups based on the evaluation indices of the assistive device, which is a non-priority evaluation item.

Specifically, in the assistive device, the evaluation index is divided into nine types. Therefore, nine display frames 521 are shown in one line in the second display region 520. Each display frame 521 displays information on the number of past patients 92 who belong to a subgroup and information on the number of days. Since the information on the number of persons and the information on the number of days displayed on the display frame 521 are the same as the information on the display frame 511, the descriptions thereof will be omitted.

Further, the assistant 80 or the like is able to select a target subgroup. When the assistant 80 clicks the display frame 521 of a subgroup with the largest number of persons or a subgroup with a relatively large number of persons, this subgroup is set as a selected subgroup.

As described above, the goal acquisition unit 11b may acquire goals from a group of patients similar to the target patient 91. The goals may include the FIM and evaluation indices of a past patient 92 who belongs to the group of patients similar to the target patient 91 or may include one of the FIM or the evaluation indices. For example, the goal acquisition unit 11b sets FIM at the time of discharge as the long-term goal for the target patient 91. The goal acquisition unit 11b acquires FIM similar to the FIM of the past patient 92 at the time of discharge as the long-term goal for the target patient 91. The FIM in the long-term goal (e.g., 7) includes a plurality of achievement stages (1-7) and needs a long period of time for the target patient 91 to achieve. Further, the goal acquisition unit 11b sets FIM after a predetermined number of days as the short-term goal for the target patient 91. The goal acquisition unit 11b acquires FIM similar to the FIM of the past patient 92 after a predetermined number of days as the short-term goal for the target patient 91. The FIM in the short-term goal (e.g., 3) includes a plurality of achievement stages (1-3) and needs a certain period of time for the target patient 91 to achieve.

The goal acquisition unit 11b may acquire, as the long-term goal and the short-term goal for the target patient 91, evaluation items of adaptive equipment, an assistive device or the like. The goal acquisition unit 11b may acquire the long-term goal and the short-term goal which are input by the assistant 80 or the like. The goal acquisition unit 11b may automatically acquire the long-term goal and the short-term goal from the selected group.

### <Practice Item Acquisition Unit>

The practice item acquisition unit 11c acquires practice items for achieving goals including the long-term goal and the short-term goal. The practice item acquisition unit 11c may acquire the practice items by referring to the practice items for the past patient 92 similar to the target patient 91.

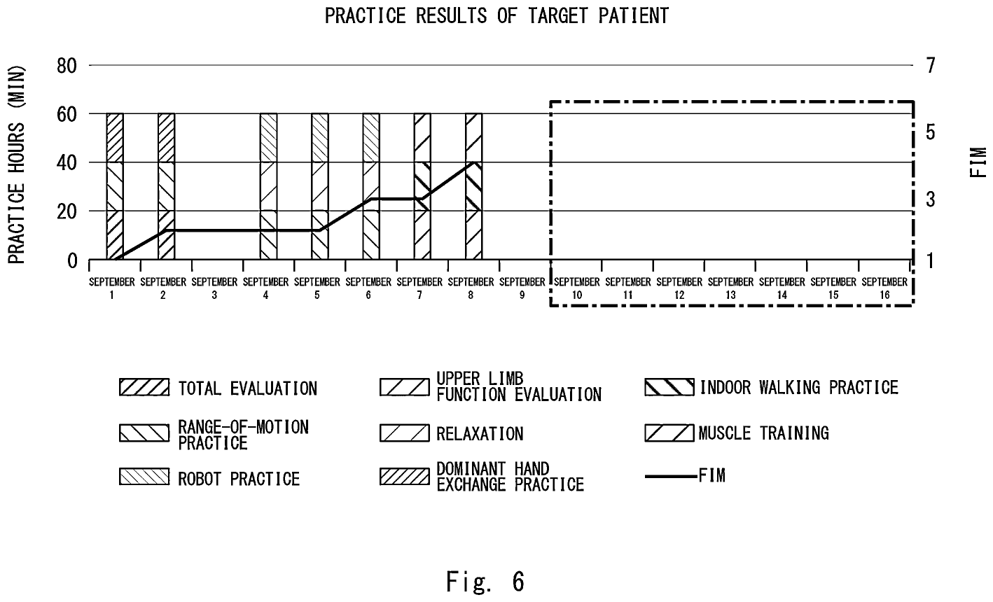

FIG. 6 is a graph illustrating the practice results and the FIM of the target patient 91 according to the first embodiment, in which the horizontal axis indicates the number of days and two vertical axes indicate the practice hours and the FIM. As shown in FIG. 6, the practice item acquisition unit 11c acquires rehabilitation patient information including the practice results and the FIM of the target patient 91. In the example shown in FIG. 6, the date of the onset of symptoms of the target patient 91 is August 31. The target patient 91 has been admitted on September 1 and started rehabilitation shortly after the admission. FIG. 6 shows practice hours of practice items and evaluation items, and the FIM recorded from September 1 to September 8.

The practice item acquisition unit 11c acquires practice results and FIM regarding other rehabilitation patients 90 (e.g., past patient 92) similar to the target patient 91. The practice item acquisition unit 11c acquires, for example, past practice results and FIM of one or more past patients 92 with the same medical condition (e.g., cerebral infarction) as the target patient 91 from the storage unit 30 or the like.

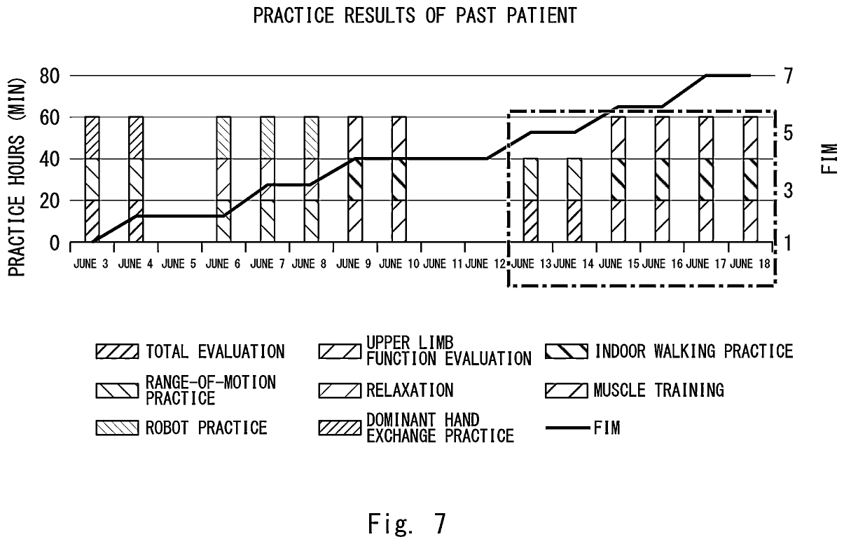

FIG. 7 is a graph illustrating the practice results and the FIM of the past patient 92 according to the first embodiment, in which the horizontal axis indicates the number of days and two vertical axes indicate the practice hours and the FIM. As shown in FIG. 7, the practice item acquisition unit 11c acquires the practice results and the FIM of the past patient 92 similar to the practice results and the FIM of the target patient 91. Specifically, data in which a difference between transition including the tilt of the FIM of the target patient 91 shown in FIG. 6 and transition including the tilt of the FIM of the past patient 92 is smaller than a threshold may be extracted as similar data. As shown in FIG. 7, the past patient 92 had a cerebral infarction on June 2 (the onset of symptoms), admitted on June 3, and then started rehabilitation. Further, the past patient 92 has recovered to the same level as a healthy person (FIM: 7) on June 17 and discharged on June 18.

The practice item acquisition unit 11c acquires the practice items for the target patient 91 by matching a base time of the practice results and the FIM of the past patient 92 to the base time of the practice results and the FIM of the target patient 91. The practice item acquisition unit 11c matches, for example, the date of the onset of symptoms (June 2) of data of the results of the past patient 92 to the date of the onset of symptoms (August 31) of data of the target patient 91, to thereby acquire practice items for the target patient 91 from the practice results of the past patient 92. Specifically, the practice item acquisition unit 11c acquires practice items for the target patient 91 on September 9 and the following dates regarding which the practice results and FIM have not been entered from the practice results of the past patient 92 on June 13 and the following dates. The practice item acquisition unit 11c applies the practice results and the FIM that have been acquired to the practice items on September 9 and the following dates regarding which data have not been entered. The practice item acquisition unit 11c can acquire, from the practice plan, September 16 as the scheduled date of discharge.

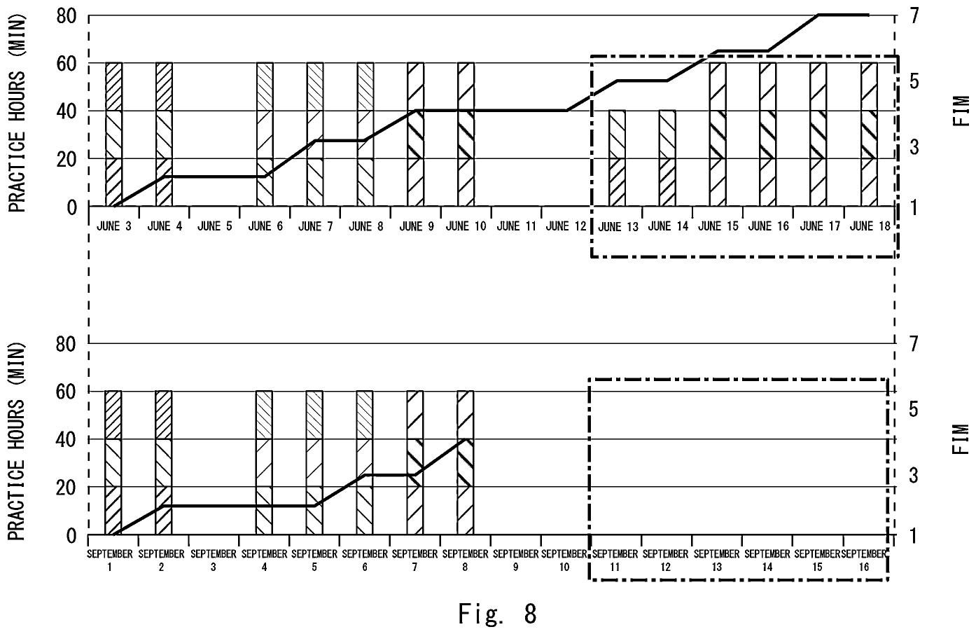

FIG. 8 is a graph illustrating the practice results and the FIM of the past patient 92 and the target patient 91 according to the first embodiment, in which the horizontal axis indicates the number of days and two vertical axes indicate the practice hours and the FIM. As shown in FIG. 8, the display unit 14 causes a display device to display the practice items for the target patient 91 (the lower diagram in FIG. 8) in such a way that they can be compared with the practice items for the past patient 92 (the upper diagram of FIG. 8). The display unit 14 shows a scheduled date of completion of practice items or a scheduled date of discharge (in the lower diagram in FIG. 8, September 16). Further, while the practice items for the target patient 91 are left blank on the display unit 14 in FIG. 8, the display unit 14 may specifically display practice items, practice hours and so on required for the target patient 91 to perform before he/she is discharged from the hospital. Alternatively, the display unit 14 may cause the display device to display the short-term goal or the long-term goal along with the practice items. The display unit 14 may simply show, instead of displaying graphs that can be compared with each other, the scheduled date of completion of practice items or the scheduled date of discharge, and practice items, practice hours and so on required for the target patient 91 to perform before he/she is discharged from the hospital.

### <Practice Result Acquisition Unit>

The practice result acquisition unit 11d acquires practice items practiced on each rehabilitation practice day as practice results. The practice items include, for example, walking practice (e.g., flat ground walking or walking on stairs), practice for increasing a range of motion, and muscle practice. Further, the practice items may include eating practice, toileting practice and so on. The practice items may be the ones in which the training apparatuses 21 and 22 are used or may be the ones in which the training apparatuses 21 and 22 are not used. The practice result acquisition unit 11d may acquire practice items as practice results as a result of input by the assistant 80 or the like.

The practice results may be associated with the name or the ID of the assigned assistant 80. The assistant 80 includes one of attributes of a physical therapist, an occupational therapist, a speech therapist, a doctor, or a nurse. The practice results may be associated with this attribute. Further, the practice results may be associated with an individual practice in which one rehabilitation patient 90 practices or a group practice in which a plurality of patients practice together. When the individual practice is performed, the assistant 80 may enter data such as practice results and the like into the rehabilitation assistance apparatus 10 after asking the rehabilitation patient 90 for the time he/she spends for each practice item. Further, the practice result acquisition unit 11d may acquire, for each practice item, the practice results for each practice item from a sensor (e.g., a wearable device) attached to the rehabilitation patient 90.

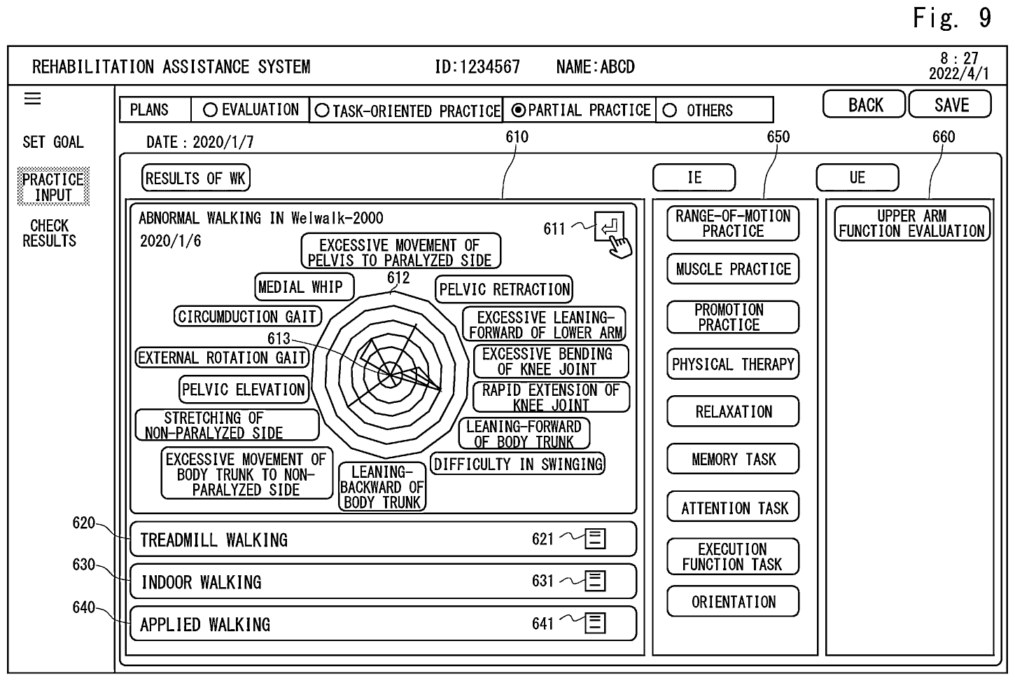

FIG. 9 is a diagram illustrating a display screen in which the display unit 14 according to the first embodiment displays practice results. As shown in FIG. 9, the display unit 14 may display practice results on the display screen. Specifically, the display screen displays a first display region 610, a second display region 620, a third display region 630, a fourth display region 640, a fifth display region 650, and a sixth display region 660.

The first display region 610 displays first information from WelWalk-2000, which is the training apparatus 21. The second display region 620 displays second information from the training apparatus 22 that provides walking training using a treadmill. The third display region 630 displays third information from the training apparatus 20 that provides indoor walking training. The fourth display region 640 displays fourth information from the training apparatus 20 that provides applied walking training.

The first display region 610, the second display region 620, the third display region 630, and the fourth display region 640 respectively include detail display buttons 611, 621, 631, and 641. By clicking the detail display buttons 611, 621, 631, and 641, details of the information are displayed. FIG. 9 shows the first information in the first display region 610 in detail. When the assistant 80 clicks the detail display button 611, the first display region 610 becomes wider. When the assistant 80 clicks the detail display button 611 once again, the first display region 610 is folded and becomes narrower.

The display unit 14 may display the first information and the second information in such a way that they are arranged close to each other (side by side). The first information and the second information may be displayed in a graph or a radar chart independently from each other. That is, the first information and the second information may be displayed in different regions (display windows) on a display screen. Alternatively, the first information and the second information may be displayed in one graph or one radar chart. The first information and the second information may be displayed in a common region (display window) of the display screen in a superimposed manner. Alternatively, the mode in which the first and second information are displayed in such a way that they are arranged close to each other is not limited to the above display mode. For example, the display unit 14 may concurrently display the first and second information on the display screen. The display unit 14 may concurrently display the first and second information in, for example, one graph on the display screen. The display unit 14 may concurrently display the first and second information in graphs different from each other. Alternatively, the display unit 14 may display the first and second information in graphs of different forms.

The first display region 610 will be described in detail. The level of achievement of training in the training apparatus 21 is evaluated in a plurality of items. Then the data regarding the plurality of items is indicated by a radar chart 612 having a circular shape or a polygonal shape.

Specifically, the training apparatus 21 detects 14 abnormal walking actions (e.g., leaning-forward of body trunk, difficulty in swinging, leaning-backward of body trunk, etc.) during training. The practice result acquisition unit 11d acquires the number of times abnormal walking actions are detected in one rehabilitation training. The display unit 14 plots the number of times of detection on a tetradecagon centered on a reference point 613. When the number of times abnormal walking actions are detected is zero, a point indicating the number of times of detection is plotted on the reference point 613. Then points indicating the number of times of detection are plotted in such a way that the distance from the reference point 613 increases as the number of times of detection increases. Therefore, the display unit 14 displays the information in such a way that the distance from the reference point 613 is changed in accordance with the level of achievement.

Further, when the rehabilitation patient 90 performs training in the training apparatus 21 a plurality of times, the display unit 14 may display the information so as to indicate a time change of the first information. For example, data acquired in the five latest training sessions may be displayed. Then the display unit 14 displays the information while changing the shapes or the colors of the points for each date of training (each date of acquisition). In this way, by displaying levels of achievement of a plurality of training sessions all at the same time, it is possible to check the degree of progress of the training. Further, the levels of achievement may be displayed in chronological order. The assistant 80 or the like may specify the date and the time when the data to be displayed has been acquired and the range of the data to be displayed.

The information indicating the level of achievement in each training apparatus 20 is displayed in each of the display regions 610-640. The number of display regions 610-640 displayed on the display screen may be changed in accordance with the number of training apparatuses 20 where training has been performed. For example, regarding a rehabilitation patient 90 who performs training only in the two training apparatuses 21 and 22, the display unit 14 may display only two display regions of the first display region 610 and the second display region 620. That is, since the information from the third training apparatus 20 and the fourth training apparatus 20 is not acquired, the display in the third display region 630 and the fourth display region 640 can be omitted. It is therefore possible to change the number of display regions for each rehabilitation patient 90.

In the second display region 620, the third display region 630, and the fourth display region 640, information in accordance with the level of achievement in each training apparatus 20 is displayed. The second display region 620 displays the second information in accordance with the level of achievement in the training apparatus 22. The third display region 630 displays the third information in accordance with the level of achievement in the third training apparatus 20, and the fourth display region 640 displays the fourth information in accordance with the level of achievement in the fourth training apparatus 20.

For example, each of the training apparatuses 20 detects abnormal actions such as abnormal walking actions of the rehabilitation patient 90. Each of the training apparatuses 20 transmits the number of times abnormal actions are detected to the rehabilitation assistance apparatus 10. The display unit 14 displays the number of times of detection as a radar chart. As a matter of course, the display unit 14 may display information other than the abnormal actions. The display unit 14 may concurrently display the details of the information obtained in two or more training apparatuses 20. Further, the display unit 14 may display information obtained in two or more training apparatuses 20 on one radar chart 612.

Training items are displayed in the fifth display region 650 and the sixth display region 660. For example, training items for improving reduced abilities (IE) are displayed in the fifth display region 650 and training items for improving upper limb functions (UE) are displayed in the sixth display region 660. Further, the rehabilitation assistance apparatus 10 may present efficient training items in accordance with the level of achievement of training performed by the rehabilitation patient 90. For example, the practice item acquisition unit 11c acquires efficient training items based on at least one of the first information or the second information. The display unit 14 may highlight efficient training items.

The training items displayed in the fifth display region 650 and the sixth display region 660 may be changed for each training apparatus 20 or may be common among a plurality of training apparatuses 20.

### <Achievement Stage Acquisition Unit>

The achievement stage acquisition unit 11e acquires the achievement stage achieved by a rehabilitation patient 90 who has practiced the practice results. The achievement stage may be indicated by predetermined evaluation items or may be indicated by FIM. When, for example, the FIM has been improved by practice results indicating the results of a walking practice carried out by the rehabilitation patient 90, the achievement stage acquisition unit 11e acquires the FIM that has been improved as the achieved achievement stage. Further, when a predetermined evaluation item has been achieved by practice results indicating the results of the walking practice carried out by the rehabilitation patient 90, the achievement stage acquisition unit 11e acquires the predetermined evaluation item as the achieved achievement stage.

The patient information acquisition unit 11a, the goal acquisition unit 11b, the practice item acquisition unit 11c, the practice result acquisition unit 11d, and the achievement stage acquisition unit 11e may be integrated as the acquisition unit 11. At least two of the patient information acquisition unit 11a, the goal acquisition unit 11b, the practice item acquisition unit 11c, the practice result acquisition unit 11d, and the achievement stage acquisition unit 11e may be integrated as the acquisition unit 11.

### <Prediction Unit>

The prediction unit 12 predicts estimated dates of goal achievement based on achieved achievement stages. The estimated dates of goal achievement include an estimated date of achievement of the long-term goal and an estimated date of achievement of the short-term goal. The prediction unit 12 may predict the estimated dates of goal achievement based on the practice results, the achievement stage, and the dates of goal achievement of other rehabilitation patients 90 (including the past patient 92) similar to the practice results of the target patient 91 to date. The dates of goal achievement include the date of achievement of the short-term goal when the other rehabilitation patient 90 has achieved the short-term goal and the date of achievement of the long-term goal when the other rehabilitation patient 90 has achieved the long-term goal.

The prediction unit 12 may further predict each estimated date of goal achievement based on the practice results associated with each assistant 80. Accordingly, it is possible to find an assistant 80 who is suitable for advancing the estimated dates of goal achievement of the rehabilitation patient 90.

### <Storage Unit>

The storage unit 13 or the like (at least one of the storage unit 13 or the database DB) stores the long-term goal and the short-term goal acquired by the goal acquisition unit 11b, the practice results acquired by the practice result acquisition unit 11d, the achievement stage acquired by the achievement stage acquisition unit 11e, and the estimated dates of goal achievement predicted by the prediction unit 12. The storage unit 13 or the like may store the long-term goal, the short-term goal, the practice results, the achievement stage, the date of achievement of the short-term goal and the date of achievement of the long-term goal of the other rehabilitation patient 90 (including the past patient 92).

The storage unit 13 or the like may store an assistant who has assisted the rehabilitation patient 90 in association with the practice results when the rehabilitation patient 90 has practiced practice items for recovering functions of his/her physical actions. The assistant 80 includes at least one of attributes of a physical therapist, an occupational therapist, a speech therapist, a doctor, or a nurse. The storage unit 13 or the like may store each attribute in association with the practice results. It is therefore possible to let the assistant 80 who is suitable for a practice item to assist this practice item, thereby improving the achievement stage of the rehabilitation patient 90. It is further possible to let the assistant 80 assist a practice item suitable for each attribute, thereby improving the achievement stage of the rehabilitation patient 90.

Further, the storage unit 13 or the like may store an individual practice carried out by one person or a group practice carried out by a plurality of persons in association with the practice results when the rehabilitation patient 90 practices practice items for recovering functions of his/her physical actions. Some practice items that the rehabilitation patient 90 practices are preferably carried out in the form of an individual practice and other practice items are preferably carried out in the form of a group practice. Accordingly, by comparing an achievement stage which is based on the practice results of an individual practice with an achievement stage which is based on the practice results of a group practice, a more suitable practice method can be selected.

Further, the storage unit 13 or the like may store rehabilitation patient information of the rehabilitation patient 90. The storage unit 13 or the like may store, for each rehabilitation patient 90, the evaluation indicates of all the evaluation items. As a matter of course, regarding some evaluation items, evaluation indices may be left blank in the storage unit 13 or the like. The storage unit 13 or the like may update the evaluation indices in accordance with the state of recovery of the rehabilitation patient 90. That is, the storage unit 13 or the like stores the latest evaluation indices. The evaluation indices are associated with the date of the acquisition when the data has been acquired. The evaluation indices may be associated with the number of days passed since the date of the onset of symptoms and the date of the start of the rehabilitation. The storage unit 13 or the like may store, besides the date of acquisition, the time of the acquisition etc.

The storage unit 13 or the like may store initial (at the time of the onset of symptoms) evaluation indices or may store evaluation indices for each predetermined period of time after the date of the onset of symptoms. The storage unit 13 or the like may store evaluation indices in accordance with the number of days since the rehabilitation is started in time series. The evaluation indices of the evaluation items may be input using the terminal 31 or may be automatically acquired by the training apparatus 20.

The storage unit 13 or the like includes a non-volatile memory such as a hard disc drive, a flash memory, an Erasable Programmable Read Only Memory (EPROM) or a Solid State Drive (SSD). The storage unit 13 or the like may include a database DB that stores information regarding a plurality of rehabilitation patients 90. The storage unit 13 may store, for each rehabilitation patient 90, information regarding the rehabilitation patient 90.

### <Display Unit>

The display unit 14 causes, for example, a display device such as a display to display the long-term goal, the short-term goal, the practice items, the practice results, the achievement stage, the estimated date of achievement of the long-term goal, the estimated date of achievement of the short-term goal, and so on for the assistant 80 or the like. The display unit 14 may cause the display device to display, for example, the practice results, the achievement stage, the estimated dates of goal achievement and so on in a superimposed manner. The display unit 14 may extract the practice results, the achievement stage, the estimated dates of goal achievement, and so on associated with a specific assistant 80 and cause the display device to display the extracted information.

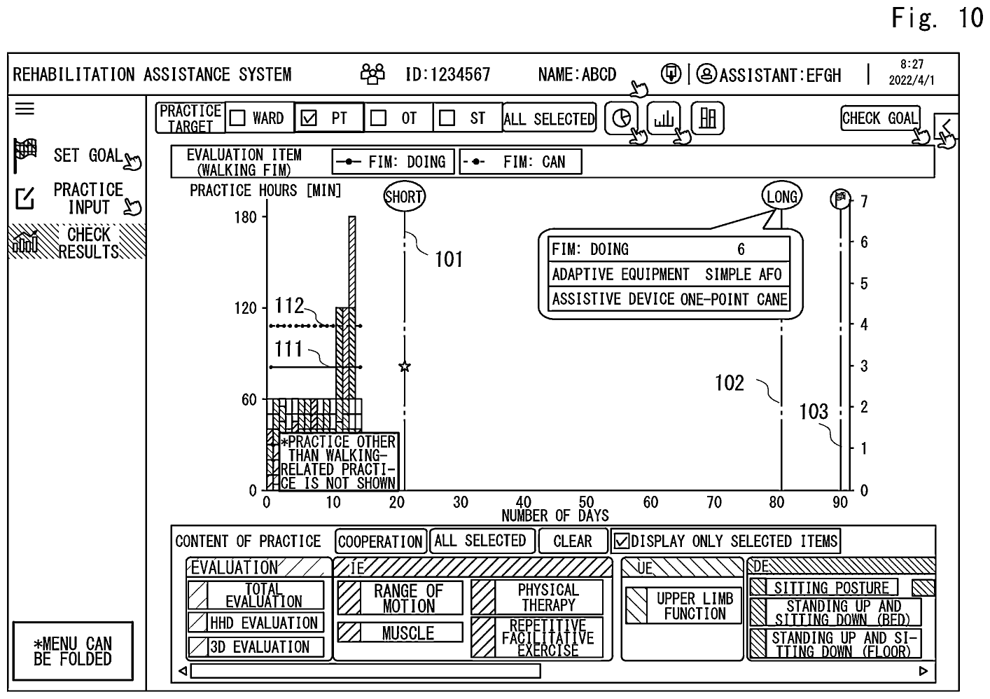

FIG. 10 is a diagram illustrating a display screen in which the display unit 14 displays the short-term goal, the long-term goal, the practice results, and the achievement stage according to the first embodiment. As shown in FIG. 10, the display unit 14 displays, in the center part of the display screen, a graph in which the horizontal axis indicates the number of days and two vertical axes indicate the practice hours and the FIM. The vertical axis on the left side of the graph indicates the practice hours, and the vertical axis on the right side of the graph indicates the FIM. Accordingly, the assistant 80 or the like can check the practice results, the FIM, and the like which are arranged close to each other.

The practice results and the FIM may set the 0-th day on the horizontal axis as the date of the onset of symptoms. The date of the onset of symptoms means the date when symptoms began to appear. The 0-th day on the horizontal axis may be changed to the date of admission or the date of the start of the rehabilitation. The assistant 80 or the like may change the 0-th day on the horizontal axis to the date of admission, the date of the start of the rehabilitation, or the like, not the date of the onset of symptoms, by operating various buttons on the display screen.

The display unit 14 may visualize the practice results of the rehabilitation patient 90 on the display screen by using stacked bar graphs or line graphs. The display unit 14 may display practice hours of the rehabilitation patient 90 for each practice item recorded by the assistant 80 as the practice results. In FIG. 10, the display unit 14 displays a line 101 indicating the short-term goal at the point where the number of practice days in the horizontal axis is 20 days. Further, the display unit 14 displays a line 102 indicating the long-term goal at the point where the number of practice days in the horizontal axis is 80 days. The display unit 14 displays a line 103 indicating the scheduled date of discharge at the point where the number of practice days in the horizontal axis is 90 days.

The line 101 of the short-term goal and the line 102 of the long-term goal may be acquired by the aforementioned goal acquisition unit 11b. The line 103 of the scheduled date of discharge may be acquired by the aforementioned practice item acquisition unit 11c. Note that the respective lines 101-103 of the short-term goal, the long-term goal, and the scheduled date of discharge may be arbitrarily set by the assistant 80 or the like. The practice items may be, for example, but not limited to, walking practice (e.g., flat ground walking or walking on stairs), eating practice, or toileting practice.

Further, as shown in FIG. 10, the display unit 14 displays “FIM: doing” 111 and “FIM: can do” 112 in association with data such as practice results.

“FIM: doing” indicates the degree to which the rehabilitation patient 90 performs a practice item in his/her daily life. For example, regarding a walking practice, a state in which the rehabilitation patient 90 can barely walk without a cane but is walking with a cane in his/her daily life is expressed as “the rehabilitation patient can walk with a cane” in “FIM: doing”. On the other hand, the same state is expressed as “the rehabilitation patient can walk without a cane” in “FIM: can do”. “FIM: doing” may be acquired for each practice item by the assistant 80 who regularly (e.g., once in a month) observes the state of the rehabilitation patient 90. “FIM: can do” may be acquired for each practice item by the assistant 80 who observes the patient in daily life. Further, “FIM: doing” is acquired by the assistant 80 at the time of both admission and discharge, and the difference between them may be used to evaluate rehabilitation results.

In the viewpoint of recovery, the value of “FIM: doing” and the value of “FIM: can do” preferably match each other. Accordingly, the assistant 80 preferably take some measures for the patient to minimize the difference between these values as much as possible.

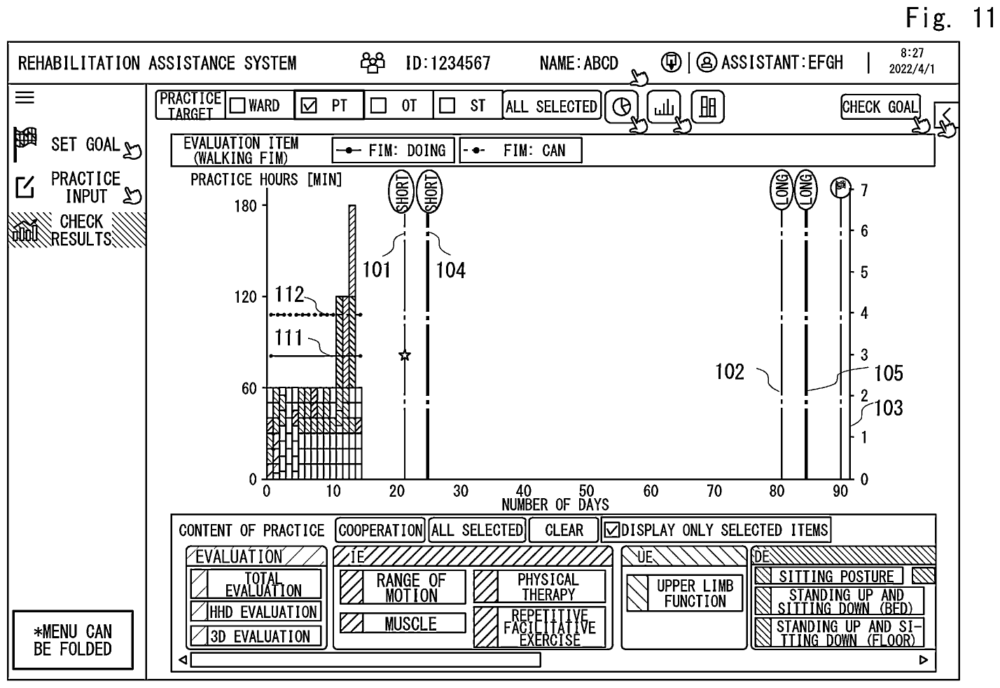

FIG. 11 is a diagram illustrating a display screen in which the display unit 14 displays the short-term goal, the long-term goal, the practice results, the achievement stage, and the estimated dates of goal achievement according to the first embodiment. As shown in FIG. 11, the display unit 14 may cause the display device to display the estimated dates of goal achievement including the estimated date of achievement of the short-term goal (line 104) and the estimated date of achievement of the long-term goal (line 105) predicted by the prediction unit 12 in a superimposed manner. It is therefore possible to intuitively recognize the difference between the short-term goal and the long-term goal that have been planned. Accordingly, the assistant 80 or the like is able to easily know the progress state of the rehabilitation in view of the short-term goal and the long-term goal.

Specifically, when the gap between the line 101 of the short-term goal and the line 104 of the estimated date of achievement of the short-term goal is large, it indicates that there are some problem in the practice results for the short-term goal. In this case, the display unit 14 may display an alert to inform the assistant 80 or the like of information indicating that it is possible that the rehabilitation patient 90 may not be able to achieve the short-term goal. Therefore, the assistant 80 or the like may review the practice items to help the patient to improve his/her achievement stage. For example, the assistant 80 or the like may cause the practice item acquisition unit 11c to acquire practice items that may help the patient to improve the achievement stage. The practice item acquisition unit 11c acquires new practice items based on the practice results, the achievement stage, and the dates of goal achievement of the other rehabilitation patient 90, and the estimated dates of goal achievement predicted by the prediction unit 12.

On the other hand, when the gap between the line 101 of the short-term goal and the line 104 of the estimated date of achievement of the short-term goal is small, this means that the practice results for the short-term goal are satisfactory. In this case, the assistant 80 or the like can maintain this pace. The same holds true for the line 102 of the long-term goal and the line 105 of the estimated date of achievement of the long-term goal. Accordingly, the assistant 80 or the like is able to know the progress of the goal achievement based on the practice results and the achievement stage and change the practice items. Further, by knowing the progress of the goal achievement, it is possible to improve the motivation for the rehabilitation.

The display unit 14 may cause the display screen to display the assistant 80 associated with the practice results. For example, the display unit 14 may cause the display screen to display the assistant 80 associated with the walking practice. Further, the assistant 80 or the like may extract practice results associated with the specific assistant 80 and cause the display device to display the extracted practice results by selecting the name or the like of the assistant 80 on the display screen. The display unit 14 may display data such as practice results associated with the assigned assistant 80 in such a way that they can be distinguished from each other using different colors. The display unit 14 may display assistants 80 in such a way that they can be distinguished from each other by their attributes. Further, the display unit 14 may display an individual practice in which one rehabilitation patient 90 practices and a group practice in which a plurality of patients practice together in such a way that they can be distinguished from each other.

### <Rehabilitation Assistance Method>

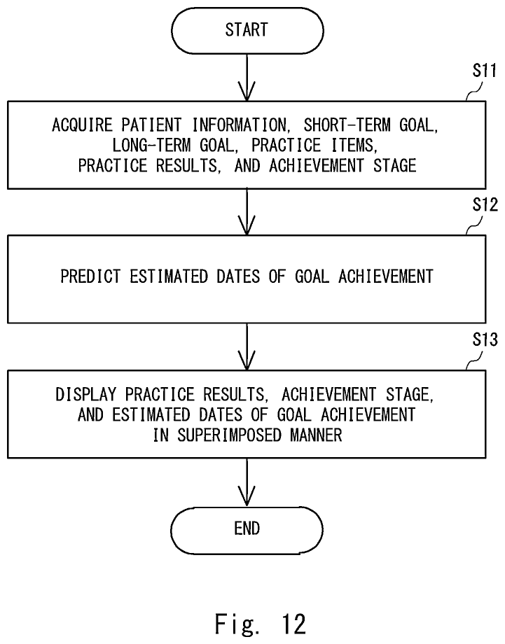

Next, a rehabilitation assistance method according to this embodiment will be described. FIG. 12 is a flowchart illustrating a rehabilitation assistance method according to the first embodiment.

As shown in Step S11 in FIG. 12, patient information, a short-term goal, a long-term goal, practice items, practice results, an achievement stage, and so on are acquired. Specifically, for example, the patient information acquisition unit 11a acquires rehabilitation patient information stored in the storage unit 13.

Before Step S11, as the rehabilitation patient information, a step of storing patient information, a short-term goal, a long-term goal, practice items, practice results, an achievement stage, a date of achievement of the short-term goal, and a date of achievement of the long-term goal, and so on of the other rehabilitation patient 90 in the storage unit 13 may be further provided. Then, the patient information acquisition unit 11a may acquire rehabilitation patient information on the other rehabilitation patient 90 from the storage unit 13.

The goal acquisition unit 11b acquires the short-term goal and the long-term goal. Specifically, the goal acquisition unit 11b acquires a long-term goal to be achieved over a long period of time and a short-term goal to be achieved in a short period of time for recovering functions of predetermined physical actions in the rehabilitation patient 90. The goal acquisition unit 11b may acquire the short-term goal and the long-term goal by referring to rehabilitation patient information of the other rehabilitation patient 90.

The practice item acquisition unit 11c acquires practice items. The practice item acquisition unit 11c may acquire practice items based on the practice items, practice results, and the achievement stage of the other rehabilitation patient 90 similar to the rehabilitation patient information, the practice results, and the achievement stage of the target patient 91.

The practice result acquisition unit 11d acquires the practice results. Specifically, the practice result acquisition unit 11d acquires practice items practiced on each practice day as the practice results. The achievement stage acquisition unit 11e acquires the achievement stage achieved by the practice results.

Next, as shown in Step S12, the estimated dates of goal achievement are predicted. Specifically, the prediction unit 12 predicts, based on the achieved achievement stage, the estimated dates of goal achievement including the estimated date of achievement of the long-term goal and the estimated date of achievement of the short-term goal. When the estimated dates of goal achievement are predicted, the prediction unit 12 may predict the estimated dates of goal achievement based on practice results, an achievement stage, and dates of goal achievement of the other rehabilitation patient 90 similar to the practice results and the achievement stage of the target patient 91. Further, the prediction unit 12 may predict each estimated date of goal achievement based on the practice results and the achievement stage associated with each assistant 80.

Note that the practiced practice results, the achieved achievement stage, and the estimated dates of goal achievement that have been predicted may be stored in the storage unit 13. Specifically, the acquisition unit 11 and the prediction unit 12 may store each of the information items that have been acquired and the estimated dates of goal achievement that have been predicted in the storage unit 13. When each of the information items that have been acquired and the estimated dates of goal achievement that have been predicted are stored in the storage unit 13, the assistant 80 who has assisted the rehabilitation patient 90 may be stored in association with practice results. Further, when the rehabilitation patient 90 practices the practice items, an individual practice carried out by one person or a group practice carried out by a plurality of persons may be stored in association with the practice results.

Next, as shown in Step S13, the practice results, the achievement stage, and so on, and the estimated dates of goal achievement are displayed on the display device in a superimposed manner. When the display unit 14 causes the display device to display the practice results, the achievement stage, and so on, and the estimated dates of goal achievement in a superimposed manner, the display unit 14 may extract practice results associated with the specific assistant 80 and cause the display device to display the extracted practice results. Accordingly, the rehabilitation assistance apparatus 10 may cause the display device to display various kinds of data of the rehabilitation patient 90.

Next, effects of this embodiment will be described. The rehabilitation assistance apparatus 10 according to this embodiment causes the display device to display the goal for recovering functions of predetermined physical actions of the rehabilitation patient 90, the practice results, the achievement stage, and the estimated dates of goal achievement in a superimposed manner. Accordingly, by displaying the planned goal along with the current practice results and the achievement stage, the rehabilitation patient 90 can intuitively check and recognize delay to the goal or the progress state. Accordingly, it is possible to maintain the motivation for rehabilitation over a long period of time from the start of training to discharge.

The prediction unit 12 predicts the estimated dates of goal achievement based on the practice results and the achievement stage of the other rehabilitation patient 90 similar to the practice results and the achievement stage of the target patient 91. It is therefore possible to improve the accuracy of the estimated dates of goal achievement.

The storage unit 13 stores the assistant 80 who has assisted the rehabilitation patient 90 in association with the practice results. Therefore, the prediction unit 12 can predict the estimated dates of goal achievement associated with each assistant 80 based on each practice result and each achievement stage associated with each assistant 80. It is therefore possible to select each practice item associated with the most suitable assistant 80 and enable the rehabilitation patient 90 to practice with this assistant 80 in order to advance the estimated dates of goal achievement.

Further, the storage unit 13 stores an individual practice carried out by one person or a group practice carried out by a plurality of persons in association with practice results. Accordingly, the prediction unit 12 is able to predict the estimated dates of goal achievement based on each practice result and each achievement stage associated with the individual practice or the group practice. It is therefore possible to select each suitable practice item of the individual practice or the group practice and cause the rehabilitation patient 90 to carry out the selected practice item in order to advance the estimated dates of goal achievement.

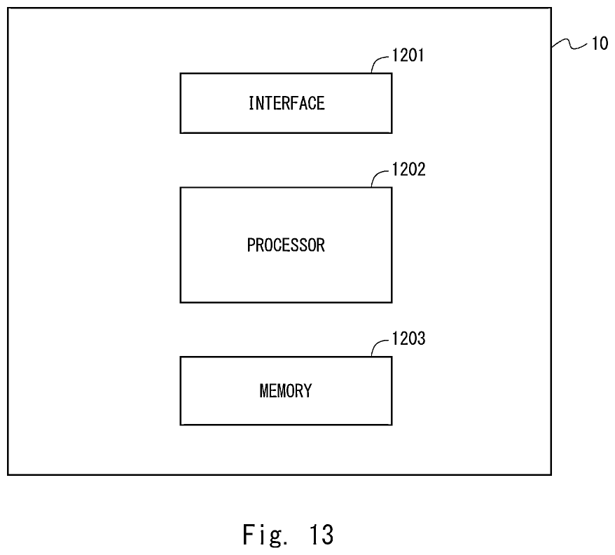

Next, a configuration of the rehabilitation assistance apparatus 10 for executing the aforementioned rehabilitation assistance method on a program will be described. FIG. 13 is a block diagram showing a configuration example of the rehabilitation assistance apparatus 10 according to the first embodiment. As shown in FIG. 13, the rehabilitation assistance apparatus 10 includes an interface 1201, a processor 1202, and a memory 1203. The interface 1201 is used to communicate with another network node device that constitutes a communication system. The interface 1201 may be used to perform wireless communication.

The processor 1202 loads software (computer program) from the memory 1203 to execute the loaded software (computer program), thereby performing processing of the rehabilitation assistance apparatus 10 described with reference to the flowchart or the sequence in the aforementioned embodiment. The processor 1202 may be, for example, a microprocessor, a Central Processing Unit (CPU), a Micro Processing Unit (MPU), an Electronic Control Unit (ECU), a Field-Programmable Gate Array (FPGA), an Application Specific Integrated Circuit (ASIC) or the like. The processor 1202 may include a plurality of processors.

The memory 1203 is composed of a combination of a volatile memory and a non-volatile memory. The memory 1203 may include a storage located apart from the processor 1202. In this case, the processor 1202 may access the memory 1203 via an Input/Output (I/O) interface that is not shown.

The memory 1203 is used to store software modules. The processor 1202 loads these software modules from the memory 1203 and executes the loaded software modules, thereby performing processing of the rehabilitation assistance apparatus 10 described in the aforementioned embodiment. Each of the processors included in the rehabilitation assistance apparatus 10 and the like executes one or more programs including instructions for causing a computer to execute the algorithm described with reference to the drawings.

Further, a part or all of the processing in the aforementioned systems, apparatuses, terminals and the like may be implemented as a computer program. The program can be stored and provided to a computer using any type of non-transitory computer readable media. Non-transitory computer readable media include any type of tangible storage media. Examples of non-transitory computer readable media include magnetic storage media (such as flexible disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

While the embodiment of the present disclosure has been described above, the present disclosure includes any suitable modification that does not impair the object and the advantages of the present disclosure, and, furthermore, the present disclosure is not limited to the embodiments described above. Further, the configurations according to the first embodiment may be combined with each other as appropriate.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A rehabilitation assistance system comprising:
a goal acquisition unit configured to acquire a long-term goal to be achieved over a long period of time, the long-term goal having a plurality of achievement stages for recovering functions of a predetermined physical action of a rehabilitation patient, and a short-term goal to be achieved in a short period of time which is shorter than the long period of time, the short-term goal having a plurality of achievement stages;
a practice result acquisition unit configured to acquire a practice item practiced on each practice day as practice results;
an achievement stage acquisition unit configured to acquire the achievement stage achieved by the practice results;
a prediction unit configured to predict, based on the achieved achievement stage, estimated dates of goal achievement including an estimated date of achievement of the long-term goal and an estimated date of achievement of the short-term goal; and
a display unit configured to cause a display device to display the practice results, the achievement stage, and the estimated dates of goal achievement in a superimposed manner.

2. The rehabilitation assistance system according to claim 1, further comprising a storage unit configured to store the practice results, the achievement stage, and the estimated dates of goal achievement.

3. The rehabilitation assistance system according to claim 2, wherein
the storage unit stores the practice results, the achievement stage, and dates of goal achievement including the date of achievement of the short-term goal and the date of achievement of the long-term goal of another rehabilitation patient, and
the prediction unit predicts the estimated dates of goal achievement based on the practice results, the achievement stage, and the dates of goal achievement of the other rehabilitation patient.

4. The rehabilitation assistance system according to claim 3, further comprising a practice item acquisition unit configured to acquire the practice items, wherein
the practice item acquisition unit acquires the practice items based on the practice results, the achievement stage, and the dates of goal achievement of the other rehabilitation patient, and the estimated dates of goal achievement predicted by the prediction unit.

5. The rehabilitation assistance system according to claim 2, wherein the storage unit stores an assistant who has assisted the rehabilitation patient when the rehabilitation patient has performed the practice items for recovering his/her functions of the physical action in association with the practice results.

6. The rehabilitation assistance system according to claim 5, wherein
the assistant comprises an attribute of at least one of a physical therapist, an occupational therapist, a speech therapist, a doctor, or a nurse, and
the storage unit stores each attribute in association with the practice results.

7. The rehabilitation assistance system according to claim 5, wherein the display unit extracts the practice results associated with the specific assistant and causes the display device to display the extracted practice results.

8. The rehabilitation assistance system according to claim 5, wherein the prediction unit predicts each estimated date of goal achievement based on the practice results associated with each assistant.

9. The rehabilitation assistance system according to claim 2, wherein the storage unit stores an individual practice carried out by one person or a group practice carried out by a plurality of persons when the rehabilitation patient practices the practice items for recovering his/her functions of the physical action in association with the practice results.

10. A rehabilitation assistance method comprising:
acquiring a long-term goal to be achieved over a long period of time, the long-term goal having a plurality of achievement stages for recovering functions of a predetermined physical action of a rehabilitation patient, and a short-term goal to be achieved in a short period of time which is shorter than the long period of time, the short-term goal having a plurality of achievement stages;
acquiring practice items practiced on each practice day as practice results;
acquiring the achievement stage achieved by the practice results;
predicting estimated dates of goal achievement including an estimated date of achievement of the long-term goal and an estimated date of achievement of the short-term goal based on the achieved achievement stage; and
causing a display device to display the practice results, the achievement stage, and the estimated dates of goal achievement in a superimposed manner.

11. The rehabilitation assistance method according to claim 10, comprising storing the practice results, the achievement stage, and the estimated dates of goal achievement in a storage unit.

12. The rehabilitation assistance method according to claim 11, comprising:
storing the practice results, the achievement stage, and dates of goal achievement including the date of achievement of the short-term goal and the date of achievement of the long-term goal of another rehabilitation patient; and
predicting, when the estimated dates of goal achievement are predicted, the estimated dates of goal achievement based on the practice results, the achievement stage, and the dates of goal achievement of the other rehabilitation patient.

13. The rehabilitation assistance method according to claim 12, comprising:
acquiring the practice items; and
acquiring, when the practice items are acquired, the practice items based on the practice results, the achievement stage, and the dates of goal achievement of the other rehabilitation patient, and the estimated dates of goal achievement that have been predicted.

14. The rehabilitation assistance method according to claim 11, comprising storing, when the practice results, the achievement stage, and the estimated dates of goal achievement are stored, an assistant who has assisted the rehabilitation patient when the rehabilitation patient has performed the practice items for recovering his/her functions of the physical action in association with the practice results.

15. The rehabilitation assistance method according to claim 14, wherein
the assistant comprises an attribute of at least one of a physical therapist, an occupational therapist, a speech therapist, a doctor, or a nurse, and
when the practice results, the achievement stage, and the estimated dates of goal achievement are stored, each attribute is stored in association with the practice results.

16. The rehabilitation assistance method according to claim 14, wherein, when the practice results, the achievement stage, and the estimated dates of goal achievement are displayed on the display device in a superimposed manner, the practice results associated with the specific assistant may be extracted and the display device may display these extracted practice results.

17. The rehabilitation assistance method according to claim 14, comprising predicting, when the estimated dates of goal achievement are predicted, an each estimated date of goal achievement based on the practice results associated with each assistant.

18. The rehabilitation assistance method according to claim 11, comprising storing, when the practice results, the achievement stage, and the estimated dates of goal achievement are stored, an individual practice carried out by one person or a group practice carried out by a plurality of persons when the rehabilitation patient practices the practice items for recovering his/her functions of the physical action in association with the practice results.

19. A non-transitory computer readable medium storing a control program for causing a computer to execute processing of:
acquiring a long-term goal to be achieved over a long period of time, the long-term goal having a plurality of achievement stages for recovering functions of a predetermined physical action of a rehabilitation patient, and a short-term goal to be achieved in a short period of time which is shorter than the long period 5 of time, the short-term goal having a plurality of achievement stages;
acquiring practice items practiced on each practice day as practice results;
acquiring the achievement stage achieved by the practice results;
predicting estimated dates of goal achievement including an estimated date of achievement of the long-term goal and an estimated date of achievement of the short-term goal based on the achieved achievement stage; and
causing a display device to display the practice results, the achievement stage, and the estimated dates of goal achievement in a superimposed manner.
